(19) United States
(12) Patent Application Publication
(10) Pub. No.: US 2017/0181880 A1
(43) Pub. Date: Jun. 29, 2017

(54) AUTOMATED POSTURE CORRECTION SYSTEM

(71) Applicants: Nazmi Peyman, Glen Allen, VA (US); Omid Souresrafil, Maple Grove, MN (US); Ehson Souresrafil, Maple Grove, MN (US)

(72) Inventors: Nazmi Peyman, Glen Allen, VA (US); Omid Souresrafil, Maple Grove, MN (US); Ehson Souresrafil, Maple Grove, MN (US)

(21) Appl. No.: 15/458,481

(22) Filed: Mar. 14, 2017

Related U.S. Application Data
(60) Provisional application No. 62/331,643, filed on May 4, 2016.

(51) Int. Cl. A61F 5/02; A61F 5/32; A61F 5/34

(52) U.S. Cl. CPC A61F 5/024; A61F 5/34; A61F 5/32

(57) ABSTRACT

The automated posture correction system comprises a spinal brace and an electronic device. The spinal brace configured to be worn by the user comprises inflatable elements and at least one actuator. The inflatable elements, positioned along the length of the predetermined sections of the spinal area of the user, comprise sensors for detecting one or more parameters of the spinal area. The electronic device receives sensor data variables generated by the sensors based on the detected parameters via a communications network. The electronic device compares the parameters with a set of preset reference values stored in memory unit. The electronic device actuates the actuator to inflate or deflate the inflatable elements based on the sensor data variables differing from the set of preset reference values. The inflatable elements selectively apply force on the predetermined sections of the spinal area for dynamically improving the posture of the user.

### TECHNICAL FIELD OF THE INVENTION

The invention disclosed herein generally relates to spinal care systems. More particularly, the invention relates to an automated posture correction system for dynamically improving a posture of a user by selective application of force on predetermined sections of a spinal area of the user.

### BACKGROUND

Posture, in general, refers to the positioning of the body against gravity while standing, sitting, or walking. An incorrect posture, over time, contributes to a majority of spine related aches or injuries. Since posture is a characteristic unique to an individual, there is no single overarching solution to the problem. Spinal pain is a significant medical problem that effects a large percentage of the population. Conventionally, various treatment methods have been implemented to alleviate or prevent pain in the cervical, thoracic, or lumber region of the spine. One of these treatment methods has been the use of devices, for example, braces, supportive and restrictive corsets, etc. These devices limit the range of motion of the spine in a passive or active way. Active movement of the spinal region refers to the patient using his/her muscles to move the spinal region, for example, flexion/extension of side bending. Alternately, passive movement of the spinal region refers to movement of the spinal region facilitated by an external force, for example, gravity, a therapist, etc. Incidentally, braces used presently are either flexible and do not restrict patient's motion to a significant degree or are made of non-flexible material where a patient's spine would not be able to move actively. A system, which dynamically adapts and supports a spinal region of a user for improving the posture of the user, is required.

Additionally, an orientation of the spinal region is dependent on a multitude of other factors, for example, age, gender, etc. For individuals of a particular age group, the corrective action or method used for posture correction depends also on the existing health condition of the individual/s. In some cases, due to the lack of strength in the spinal muscles of a patient, it is highly disadvantageous to travel multiple times to consult a physician. With the advent of remote health care methods, it is now possible for a patient to consult a physician remotely from the comforts of his/her home. However, with regard to posture correction, there is a lack of a system, which allows a physician to manipulate the posture of the patient with the help of a remote electronic device. Such an improvement in the art will permit more independence on the part of a patient to consult a remote physician and receive corrective action based on the individual's age, gender, existing health condition, etc.

Hence, there is a long felt but unresolved need for a system, which dynamically adapts and supports a spinal region of a user for improving the posture of the user. Furthermore, there is a need for a system, which allows a physician to manipulate the posture of the patient with the help of a remote electronic device.

### SUMMARY OF THE INVENTION

This summary is provided to introduce a selection of concepts in a simplified form that are further disclosed in the detailed description of the invention. This summary is not intended to identify key or essential inventive concepts of the claimed subject matter, nor is it intended for determining the scope of the claimed subject matter.

The automated posture correction system, disclosed herein, addresses the above-mentioned need for a system, which dynamically adapts and supports a spinal region of a user for improving the posture of the user. Furthermore, the invention addresses the need for a system, which allows a physician to manipulate the posture of the patient with the help of a remote electronic device. The automated posture correction system for dynamically improving a posture of a user by selective application of force on predetermined sections of a spinal area of the user comprises a spinal brace and an electronic device. The spinal brace is configured to be worn by the user comprising a plurality of inflatable elements and at least one actuator. The inflatable elements are positioned along the length of the predetermined sections of the spinal area of the user.

The inflatable elements comprise one or more sensors for detecting one or more parameters of the spinal area. The electronic device is configured to receive sensor data variables generated by the one or more sensors based on the detected one or more parameters via a communications network. Further, the electronic device is configured to compare the one or more parameters with a set of preset reference values stored in a non-transitory readable computer storage medium of the electronic device. The electronic device then actuates the at least one actuator to inflate or deflate the inflatable elements. The inflatable elements are either inflated or deflated based on the sensor data variables differing from the set of preset reference values. The inflatable elements due to their inflation or deflation selectively apply force on the predetermined sections of the spinal area for dynamically improving the posture of the user.

### BRIEF DESCRIPTION OF THE DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific methods and structures disclosed herein. The description of a method step or a structure referenced by a numeral in a drawing is applicable to the description of that method step or structure shown by that same numeral in any subsequent drawing herein.

FIG. 1 exemplarily illustrates a block diagram of an automated posture correction system.

FIG. 2 exemplarily illustrates a schematic diagram showing components of the automated posture correction system.

FIG. 3 exemplarily illustrates a spinal brace of an automated posture correction system.

FIG. 4A exemplarily illustrates a spinal brace of an automated posture correction having inflatable elements positioned perpendicular to a vertical axis.

FIG. 4B exemplarily illustrates a spinal brace of an automated posture correction having inflatable elements positioned parallel to a vertical axis.

FIG. 4C exemplarily illustrates a spinal brace of an automated posture correction having inflatable elements positioned at an inclination to a vertical axis.

FIG. 5A exemplarily illustrates a partial front elevation view of a spinal brace.

FIG. 5B exemplarily illustrates a cut away sectional view of a spinal brace about section A-A.

FIG. 6A exemplarily illustrates a spinal brace worn by a patient having an incorrect posture.

FIG. 6B exemplarily illustrates a spinal brace implementing a correction of the posture of the patient using the automated posture correction system.

### DETAILED DESCRIPTION OF THE INVENTION

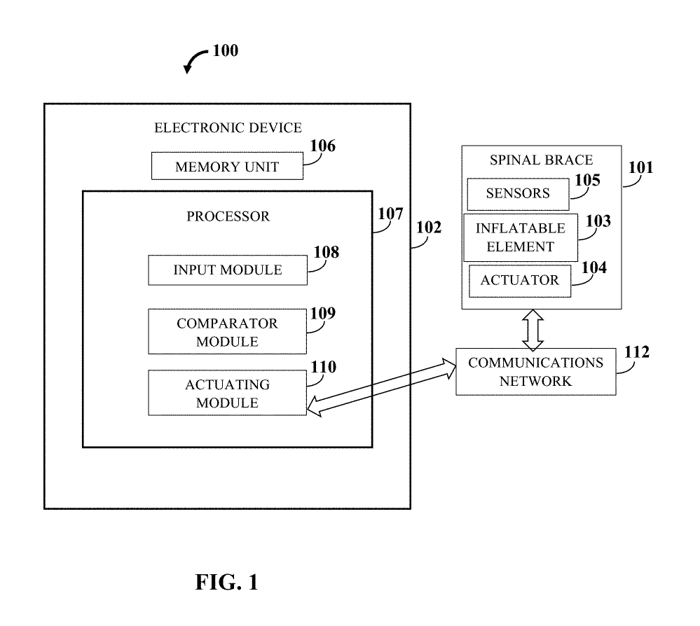

FIG. 1 exemplarily illustrates a block diagram of an automated posture correction system 100. The automated posture correction system 100 for dynamically improving a posture of a user by selective application of force on predetermined sections of a spinal area of the user comprises a spinal brace 101 and an electronic device 102. The spinal brace 101 configured to be worn by the user comprises a plurality of inflatable elements 103 and at least one actuator 104. In an embodiment, the inflatable elements 103 are positioned along the length of the predetermined sections of the spinal area of the user. Each of the inflatable elements 103 comprise one or more sensors 105 for detecting one or more parameters of the spinal area. In an embodiment, the sensors 105 are, for example, a position sensor, a pressure sensor, etc. The sensors 105 detect a position of the inflatable elements 103 relative to the spinal area of the user and a pressure within the inflatable elements 103. The sensors 105 generate sensor data variables based on the detected parameters. The parameters include, for example, a position of the inflatable element 103 relative to the spinal region of the user, pressure within the inflatable elements 103, etc. The actuator 104 either inflates or deflates the inflatable elements 103 based on the sensor data variables differing from a set of preset reference values stored in the non-transitory readable computer storage medium 106.

As used herein, a “non-transitory readable computer storage medium” refers to a memory unit of the electronic device for storing computer program instruction and other data required by the automated posture correction system 100. As such, the terms “non-transitory readable computer storage medium” and “memory unit” are used interchangeably in the specification and the drawings to represent the same component. The electronic device 102 comprises a memory unit 106 and a processor 107. The non-transitory readable computer storage medium 106 stores computer program instructions defined by modules of the automated posture correction system 100. The processor 107 is communicatively coupled to the non-transitory readable computer storage medium 106 to execute the computer program instructions defined by the modules of the automated posture correction system 100. In an embodiment, the modules of the automated posture correction system 100 comprise an input module 108, a comparator module 109, and an actuating module 110. The input module 108 is configured to receive the sensor data variables via a communications network 112. In an embodiment, the communications network 112 is, for example, a Wi-Fi network, a Bluetooth network, an NFC communications protocol, a Bluetooth Low Energy (BLE) network, etc. The comparator module 109 is configured to compare the sensor data variables with a set of preset reference values stored in the non-transitory readable computer storage medium 106.

The actuating module 110 is configured to trigger the at least one actuator 104 to one of inflate and deflate the inflatable elements 103 based on the sensor data variables differing from the set of preset reference values. The inflatable elements 103 selectively apply force on the predetermined sections of the spinal area for dynamically improving the posture of the user. As used herein, the “predetermined sections” of the spinal area refer to sections of the spinal area that the automated posture correction system 100 identifies as weak or having an incorrect inclination relative to the vertical axis. The automated posture correction system 100 does so by comparing the sensor data variables generated by the sensors 105 with a set of preset reference values. As used herein, the “set of preset reference values” comprise a reference position of the spinal area, an inclination of the spinal area relative to a vertical axis, and relative distances of the inflatable elements from the vertical axis. The set of preset reference values vary based on a gender, an age, and a health statistic of the user. The data associated with the gender, age, and the health statistic of the user is entered into the application installed on the electronic device 102. In an embodiment, the electronic device 102 is, for example, a smart phone, a laptop, a tablet, a smart watch, a personal computer, etc.

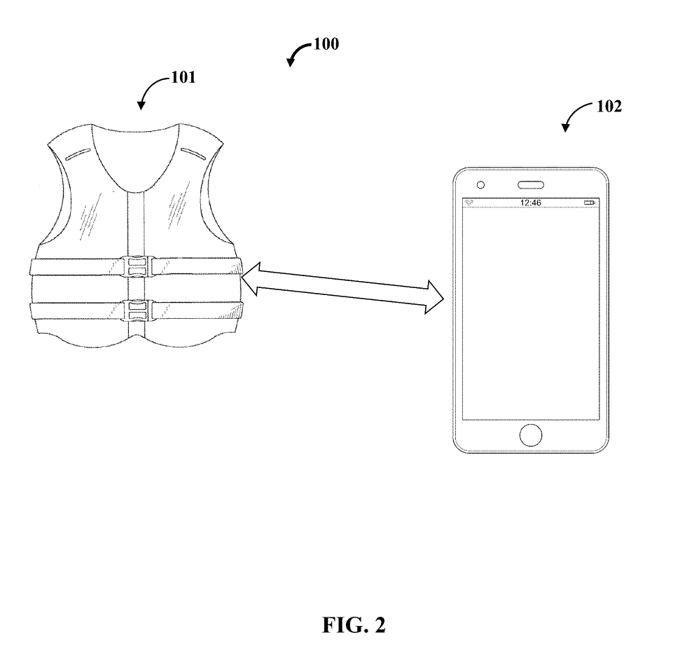

FIG. 2 exemplarily illustrates a schematic diagram showing components of the automated posture correction system 100. As disclosed in the detailed description of FIG. 1, the automated posture correction system 100 comprises a spinal brace 101 and an electronic device 102. In an embodiment, the electronic device 102 used is, for example, a smart phone device. Further, the automated posture correction system 100 uses passive and active range of motion restriction, which also corrects the posture of the user to alleviate or prevent further pain from happening. The spinal brace 101 and the electronic device 102 are programmed with the best posture for the predetermined section of the spine for a specific patient. In an embodiment, the automated posture correction system 100 uses the technology for inflation and deflation of air bags and/or rolling devices to manipulate the posture of the patient. Alternately, the automated posture correction system 100 reminds the persons in a semi-active manner to maintain proper posture throughout daily or nightly activities. The automated posture correction system 100 does not continuously restrict motion but allows the patient to maintain a proper posture. Consequently, the muscle groups of the spinal region are maintained in this posture.

Since restricting muscle-movement to maintain proper posture of the spine leads to muscle atrophy and weakness, this feature of the automated posture correction system 100 is advantageous. This would remind the individual to maintain proper posture using appropriate musculature and once the person has done so, the inflatable elements 103, exemplarily illustrated in FIG. 1, would deflate and rolling ceases and allows the muscles to take over. This would be a training tool for muscles as well. In addition, the spinal brace 101 could be set to a constant mode where the spinal brace 101 would take over and stay inflated to support the predetermined sections of the spinal region in a continuous manner. This is used for an acute phase or a phase when a restriction of the spines range of motion is desired on a continuous basis. The spinal brace 101 works with implantable, rechargeable batteries or a changeable battery of the extended range type such as a lithium battery. In an embodiment, the spinal brace 101 is plugged in if the patient plans to be stationary for an extended period. In another embodiment, remote scanning vs. touch scanning is used to assess the posture of the individual and customize the use of airbag or roller technology. This would allow for optimum posture correction on a custom, individual basis.

A set of reference values for an optimum posture is pre-programmed in the electronic device 102 based on a patient's height, weight, etc., or as assessed by a specialist. The specialist then populates the information on the electronic device 102. The inflatable elements 103, exemplarily illustrated in FIG. 1, and/or rollers will gently push patient's segment of spine or body section into the direction of proper posture. In an embodiment, verbal commands can also be used to actuate the inflatable elements 103, but the main modality is an actual physical push on the predetermined sections of the spinal area. In an embodiment, the electronic device 102 will either constantly monitor a variety of angles or react to pressure sensed on its sensors 105, exemplarily illustrated in FIG. 1. For example if one's spine is in excessive kyphosis, the sensors 105 detect abnormal curvature and angles. In such a case, the spinal area will be pressed upon differently than when the person's spine was upright. The processor 107 of the electronic device 102 actuates the actuator 104 to inflate the required inflatable elements and/or rollers to correct the angles and curvatures by pushing on the predetermined sections of the spinal area or body segment as exemplarily illustrated in FIG. 1.

In an embodiment, the electronic device 102 is capable of communicating wirelessly with the wearable spinal brace 101 via the communications network 112 as exemplarily illustrated in FIG. 1. In an embodiment, the electronic device 102 allows for the programming of passive and active range of motion restriction and corrects the posture of the user to alleviate or prevent further pain from happening. Additionally, the spinal brace 101 is programmable via the electronic device 102 to obtain the best posture for the particular part of the spine for that particular person. In an embodiment, rechargeable batteries or a changeable battery with extended range, for example, a lithium battery, powers both the spinal brace 101 and the electronic device 102. The spinal brace 101 could also be plugged in if the patient plans to be stationary for an extended period.

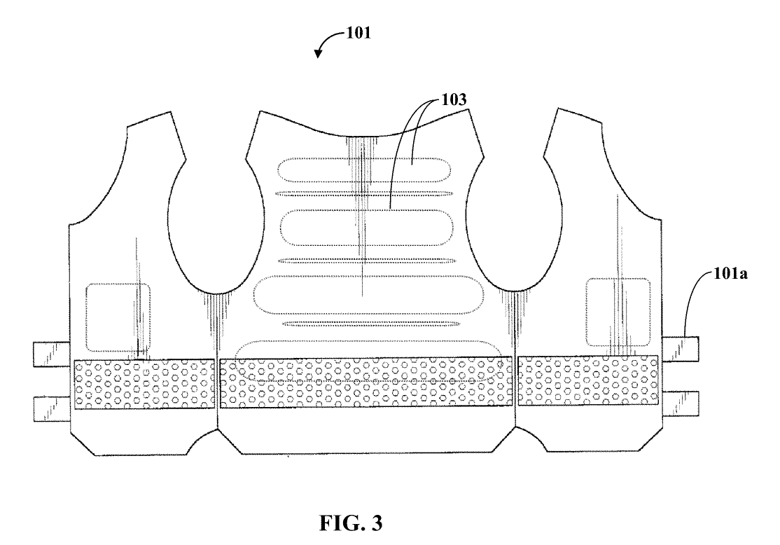

FIG. 3 exemplarily illustrates a spinal brace 101 of an automated posture correction system 100. In an embodiment, the spinal brace 101 is configured as a wearable jacket as exemplarily illustrated in FIG. 3. The spinal brace 101 comprises inflatable elements 103 embedded within the interior of the jacket. The spinal brace 101 comprises fastening elements 101a, which are used to fasten the spinal brace 101 to a torso of a user. In an embodiment, the fastening elements 101a are, for example, snap fasteners, hook and loop fasteners, clip fasteners, etc.

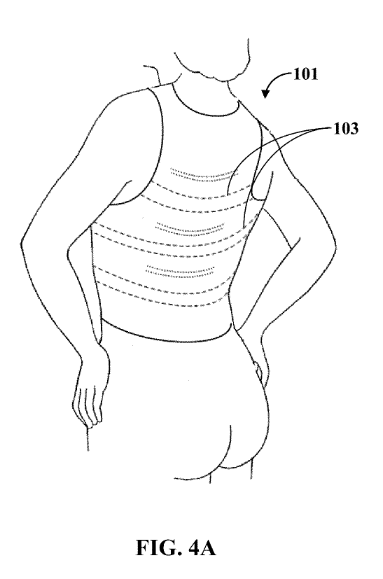

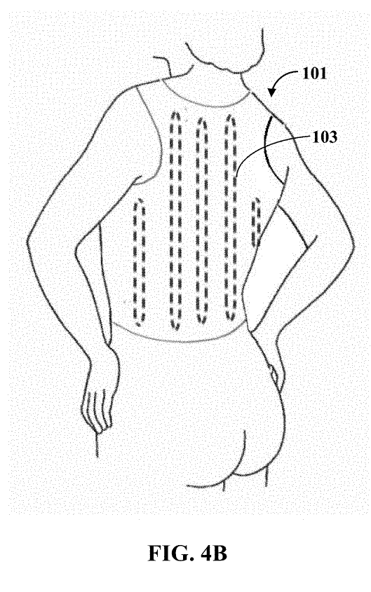

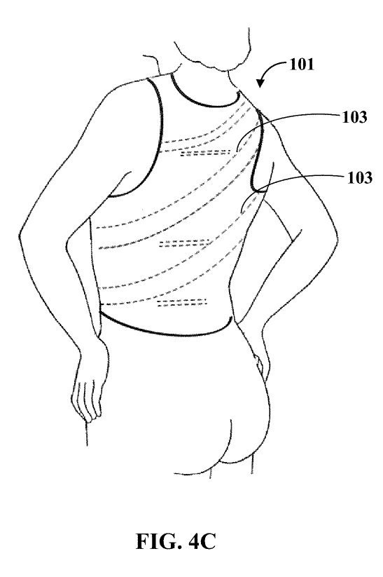

FIG. 4A exemplarily illustrates a spinal brace 101 of an automated posture correction system 100 having inflatable elements 103 positioned perpendicular to a vertical axis. FIG. 4B exemplarily illustrates a spinal brace 101 of an automated posture correction system 100 having inflatable elements 103 positioned parallel to a vertical axis. FIG. 4C exemplarily illustrates a spinal brace 101 of an automated posture correction system 100 having inflatable elements 103 positioned at an inclination to a vertical axis.

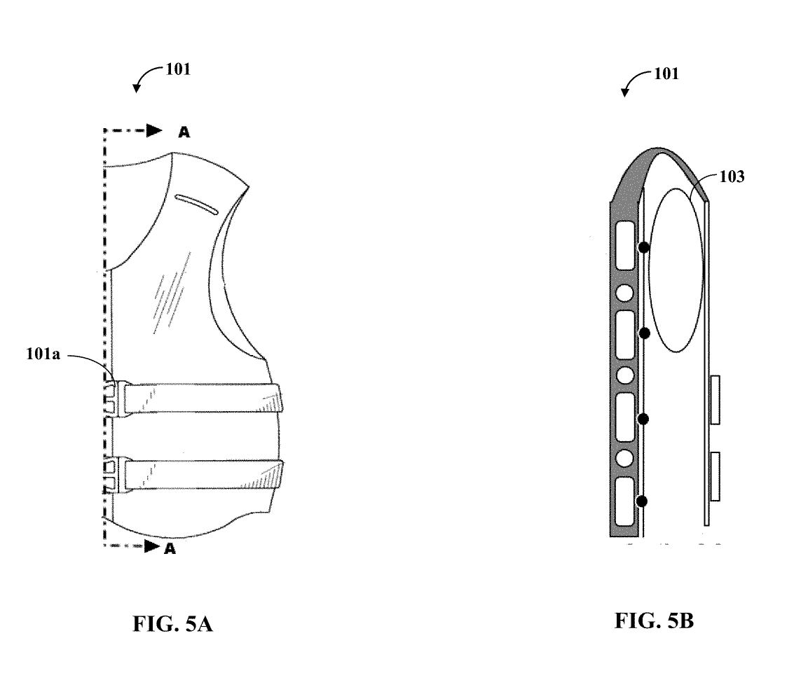

FIG. 5A exemplarily illustrates a partial front elevation view of a spinal brace 101. In an embodiment, the spinal brace 101 comprises clip fasteners 101a to strap the spinal brace 101 on a torso of a patient/user.

FIG. 5B exemplarily illustrates a cut away sectional view of a spinal brace 101 about section A-A exemplarily illustrated in FIG. 5A. As disclosed in the detailed descriptions of FIGS. 1-2, the spinal brace comprises multiple inflatable elements 103 embedded within the spinal brace 101. The respective inflatable element 103 within the spinal brace 101 is inflated or deflated to selectively apply force to the predetermined section of the spinal area.

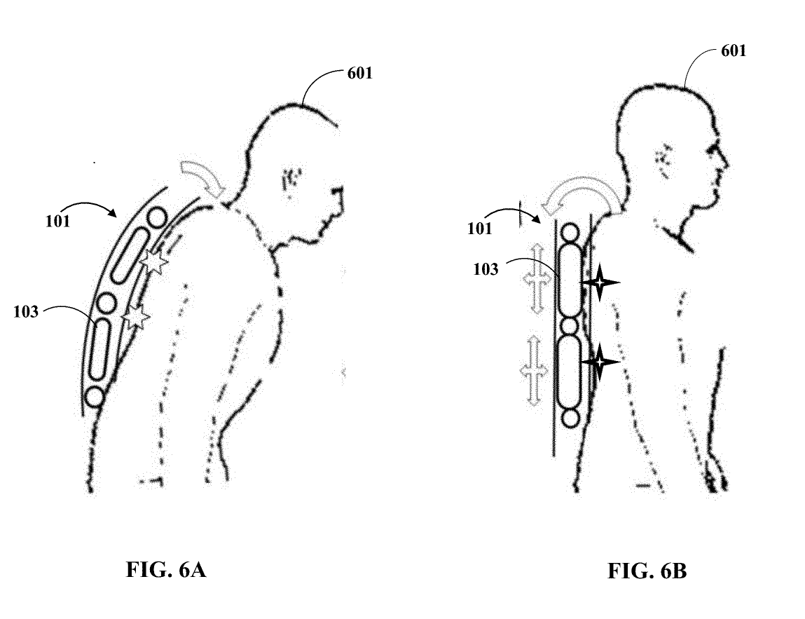

FIG. 6A exemplarily illustrates a spinal brace 101 worn by a patient/user 601 having an incorrect posture. The spinal brace 101 with the inflatable elements 103 in a deflated state conforms to the spinal curvature of the patient/user 601, as exemplarily illustrated in FIG. 6A. The sensors 105, exemplarily illustrated in FIG. 1, detect the parameters of the spinal area of the patient/user, for example, a reference position of the spinal area, an inclination of the spinal area relative to a vertical axis, and relative distances of the inflatable elements 103 from the vertical axis.

FIG. 6B exemplarily illustrates a spinal brace 101 implementing a correction of the posture of the patient/user 601 using the automated posture correction system 100, disclosed in the detailed description of FIGS. 1-2. The detected parameters are compared by the comparator module 109 of the electronic device 102 as exemplarily illustrated in FIG. 1. The automated posture correction system 100 thus identifies the weaker sections of the spinal area or incorrectly aligned position of the spinal area. The application installed on the electronic device 102 has a set of optimum reference values based on the age, gender, and existing health condition of the patient/user. The electronic device 102 then actuates the actuator to inflate the inflatable elements 103 to selectively apply force on the predetermined weak or incorrectly aligned sections of the spinal area for dynamically improving the posture of the user as exemplarily illustrated in FIG. 6B.

The foregoing examples have been provided merely for the purpose of explanation and are in no way to be construed as limiting of the automated posture correction system 100, disclosed herein. While the automated posture correction system 100 has been described with reference to various embodiments, it is understood that the words, which have been used herein, are words of description and illustration, rather than words of limitation. Further, although the automated posture correction system 100, has been described herein with reference to particular means, materials, and embodiments, the automated posture correction system 100 is not intended to be limited to the particulars disclosed herein; rather, the automated posture correction system 100 extends to all functionally equivalent structures, methods and uses, such as are within the scope of the appended claims. Those skilled in the art, having the benefit of the teachings of this specification, may effect numerous modifications thereto and changes may be made without departing from the scope and spirit of the automated posture correction system 100 disclosed herein in their aspects.

What is claimed is:

1. An automated posture correction system for dynamically improving a posture of a user by selective application of force on predetermined sections of a spinal area of the user, the automated posture correction system comprising:
a spinal brace configured to be worn by the user comprising a plurality of inflatable elements and at least one actuator, the inflatable elements positioned along a length of the predetermined sections of the spinal area of the user, wherein the inflatable elements comprise one or more sensors for detecting one or more parameters of the spinal area;
an electronic device configured to receive sensor data variables generated by the one or more sensors based on the detected one or more parameters via a communications network, wherein the electronic device is further configured to:
compare the one or more parameters with a set of preset reference values stored in a non-transitory readable computer storage medium of the electronic device;
actuate the at least one actuator to one of inflate and deflate the inflatable elements based on the sensor data variables differing from the set of preset reference values, wherein the inflatable elements selectively apply force on the predetermined sections of the spinal area for dynamically improving the posture of the user.

2. The automated posture correction system of claim 1, wherein the set of preset reference values comprise a reference position of the spinal area, an inclination of the spinal area relative to a vertical axis, and relative distances of the inflatable elements from the vertical axis.

3. The automated posture correction system of claim 1, wherein the set of preset reference values vary based on a gender, an age, and a health statistic of the user.

4. The automated posture correction system of claim 1, wherein the inflatable elements are positioned perpendicular to a vertical axis.

5. The automated posture correction system of claim 1, wherein the inflatable elements are positioned at an inclination to a vertical axis.

6. The automated posture correction system of claim 1, wherein the electronic device is one of a smart phone, a laptop, a tablet, a smart watch, and a personal computer.

7. The automated access management system of claim 1, wherein the sensors comprise one of a position sensor and a pressure sensor.

8. An automated posture correction system for dynamically improving a posture of a user by selective application of force on predetermined sections of a spinal area of the user, the automated posture correction system comprising:
a spinal brace configured to be worn by the user comprising:
a plurality of inflatable elements positioned along a length of the predetermined sections of the spinal area of the user, wherein each of the inflatable elements comprise one or more sensors for detecting one or more parameters of the spinal area, wherein the one or more sensors generate sensor data variables;
at least one actuator for one of inflating and deflating the inflatable elements; and
an electronic device comprising:
a non-transitory readable computer storage medium for storing computer program instructions defined by modules of the automated posture correction system;
a processor communicatively coupled to the non-transitory readable computer storage medium to execute the computer program instructions defined by the modules of the automated posture correction system, the modules comprising:
an input module configured to receive the sensor data variables via a communications network;
a comparator module configured to compare the sensor data variables with a set of preset reference values stored in the non-transitory readable computer storage medium; and
an actuating module configured to trigger the at least one actuator to one of inflate and deflate the inflatable elements based on the sensor data variables differing from the set of preset reference values, wherein the inflatable elements selectively apply force on the predetermined sections of the spinal area for dynamically improving the posture of the user.

9. The automated posture correction system of claim 8, wherein the set of preset reference values comprise a reference position of the spinal area, an inclination of the spinal area relative to a vertical axis, and relative distances of the inflatable elements from the vertical axis.

10. The automated posture correction system of claim 8, wherein the set of preset reference values vary based on a gender, an age, and a health statistic of the user.

11. The automated posture correction system of claim 8, wherein the inflatable elements are positioned perpendicular to a vertical axis.

12. The automated posture correction system of claim 8, wherein the inflatable elements are positioned at an inclination to a vertical axis.

13. The automated posture correction system of claim 8, wherein the electronic device is one of a smart phone, a laptop, a tablet, a smart watch, and a personal computer.

14. The automated access management system of claim 8, wherein the sensors comprise one of a position sensor and a pressure sensor.
